(19) United States
(12) Patent Application Publication
(10) Pub. No.: US 2002/0061927 A1
(43) Pub. Date: May 23, 2002

(54) Insulin receptor activators for the treatment of metabolic disorders induced by treatment with HIV protease inhibitors

(76) Inventors: Prasad V.V.S.V. Manchem, South San Francisco, CA (US); Robert T. Lum, Palo Alto, CA (US); Steven R. Schow, Redwood Shores, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 09/977,059

(22) Filed: Oct. 11, 2001

Related U.S. Application Data
(60) Provisional application No. 60/239,636, filed on Oct. 11, 2000.

(51) Int. Cl. A61K 31/277; A61K 31/255; A61K 31/24; A61K 31/17

(52) U.S. Cl. 514/518; 514/520; 514/535; 514/596; 514/597

(57) ABSTRACT

Methods of treating a person suffering from HIV protease inhibitor-induced metabolic disorders such as lipodystrophy, hypertriglyceridemia, insulin resistance, hyperglycemia, diabetes and ketoacidosis, comprise treatment with an insulin receptor activating compound, optionally in conjunction with insulin. In general, any insulin receptor activating compound is suitable for the practice of the invention; and preferred compounds are disclosed.

### CROSS-REFERENCE TO RELATED APPLICATION

This application claims the priority under 35 USC 119(e) of U.S. Provisional Application No. 60/239,636, filed Oct. 11, 2000, which is incorporated herein by reference.

### BACKGROUND OF THE INVENTION

(a) Field of the Invention

The present invention relates to methods, chemical compounds, and compositions for the treatment of metabolic disorders induced by treatment with HIV protease inhibitors.

(b) Description of Related Art

Treatments for HIV infection have proven very effective in controlling the ravages of the terminal stage of the infection, AIDS. The HIV protease inhibitors are an extremely important component in the drug regimens used to suppress viral load and the resulting AIDS symptoms. Unfortunately, these drugs which are required to maintain health in HIV-infected individuals also carry a significant side-effect burden. One of the recently recognized severe side-effects is HIV protease inhibitor-induced insulin resistance leading to hyperglycemia that can progress to diabetes and ultimately life threatening ketoacidosis. (Carr, A., Samaras, K., Chrisholm, D. J., and Cooper, D. A. (1998) Lancet 351 1881-1883; Carr, A., Samaras, K., Burton, S., Freund, I., Chisholm, D. J., Cooper D. A. (1998) AIDS 12, F51-F58).

In addition to insulin resistance, other related disturbances in metabolism, such as lipodystrophy and hypertriglyceridemia, are also observed in HIV protease inhibitor treated patients (Roth, V. R., Kravcik, S., Angel, J. B. (1998) Clin Infect Dis 27,65-67; Safrin, S., and Grunfeld, C., (1999) AIDS 13, 2493-2505; Carr, A., Samaras, K., Thorisdottir, A., Kaufihann, G. R., Chrishohm, D. J., and Cooper, D. A. (1999) Lancet 353, 2093-2099; Behrens, G., Dejam, A., Schmidt, H., Balks, H-J., Brabant, G., Komer, T., Stoll, M., and Schmidt, R. E. (1999) AIDS 13, F63-F70). This complex metabolic side-effect profile of these very important drugs has all the hallmark features of the insulin-resistant state referred to as Syndrome-X (Reaven, G. M. (1993) Annu. Rev. Med. 44, 121-131).

For some patients, these metabolic side-effects greatly limit the use of these life-sustaining drugs. This side-effect profile was not recognized early in the development of these drugs, but once these inhibitors entered general clinical use, this problematic side-effect manifested itself in a large percentage of the treated population. The problem appears to be a class effect in that all the currently available HIV protease inhibitor drugs demonstrate this severe effect.

The molecular origin of this phenomena was recently identified in 3T3 L1 adipocytes (Murata, H., Hruz, P. W., and Mueckler, M. (2000) The Journal of Biological Chemistry 275:27, 20251-20254). The report provided evidence that at least three of the commercialized HIV protease inhibitor drugs also inhibit the glucose transporter from localizing to the cell membrane with the subsequent inhibition of glucose uptake by these cells. This inhibition of cellular glucose transport into cells by these HIV protease inhibitors is consistent with the elevation of glucose and lipids observed in the clinic for some patients being treated with these protease inhibitor drugs.

Insulin receptor activators are known to stimulate the translocation of the glucose transporter to the cell membrane and so to stimulate the subsequent uptake of extracellular glucose. Several classes of compounds are known to act as insulin receptor activators. Insulin receptor activators include compounds of the structural classes Formulas I through VII illustrated below. Examples of compounds of formula I-II are described in WO 00/71506 and WO 01/12591. Syntheses of compounds of Formulae III and IV are described in the “Examples” section, below. Examples of compounds of Formulas V and VI are reported in U.S. Pat. No. 6,051,597, and WO 99/51225, and examples of compounds of Formula VII have been reported in U.S. Pat. Nos. 5,851,988 and 5,830,918. In addition, azo dye-like compounds have been reported by Geier et al. (U.S. Pat. No. 6,020,374).

The disclosures of these and all other documents referred to in this application are incorporated herein by reference.

### SUMMARY OF THE INVENTION

This invention includes a method of treating a metabolic disorder in a person induced by treatment of the person with an HIV protease inhibitor, comprising administering to the person a therapeutically effective dose of an insulin receptor-activating compound or a pharmaceutically acceptable salt thereof, where the compound is not insulin. In particular, this invention includes a method of such treatment where the metabolic disorder induced by treatment with an HIV protease inhibitor is selected from the group consisting of insulin resistance, hyperglycemia, diabetes, ketoacidosis, lipodystrophy, and hypertriglyceridemia. These insulin receptor-activating compounds may also be used in conjunction with insulin for such treatment.

Applicants have discovered that compounds which sensitize the insulin receptor reverse the HIV protease inhibitor side-effect of metabolic disorders such as those listed above, and can reverse the glucose uptake inhibition caused by HIV protease inhibitor drugs. Thus, applicants disclose that compounds which enhance glucose transport into cells treated with HIV protease inhibitors can provide relief from this therapy-limiting side-effect seen in protease inhibitor-treated patients. Insulin receptor activator compounds as herein disclosed can provide an effective therapy to HIV protease inhibitor-induced metabolic disorders such as insulin resistance, hyperglycemia, diabetes, ketoacidosis, lipodystrophy, and hypertriglyceridemia.

In particular, compounds of Formulas I-VII are useful for the treatment of metabolic diseases induced by treatment with an HIV protease inhibitor, and for the treatment of HIV protease inhibitor-induced insulin resistance, hyperglycemia, diabetes, ketoacidosis, lipodystrophy and hypertriglyceridemia in humans. The methods of the invention also include the co-administration of insulin in conjunction with the administration of receptor-activating compounds for such treatment. Compounds of Formula I-IV are also disclosed as being useful in treating diabetes in U.S. patent applications Nos. 09/872,763 (and Provisional Application No. 60/208,591), 09/949,165 (and Provisional Application No. 60/230,738), and 09/579,279, and PCT/US00/14644, incorporated herein by reference.

In one aspect, this invention includes a method of such treatment where the insulin receptor-activating compound is a compound of Formula I:

[chemical structure]

wherein:

R¹ and R² are substituents on the A ring and are, independently, —SO₂NR⁷₂, —C(O)NR⁷₂, —NR⁷SO₂R⁷, —NR⁷C(O)R⁷, —SO₂OR⁷, —C(O)OR⁷, —OSO₂R⁷, or —OC(O)R⁷,

R³ and R⁴ are, independently, hydrogen or lower alkyl, or R³ and R⁴ together are —(CH₂)₂—, —(CH₂)₃—, or —(CH₂)₄—,

R⁵ and R⁶ are, independently, hydrogen, lower alkyl, substituted lower alkyl, cyano, halo, nitro, —SR⁸, —C(O)R⁸, —SO₂OR⁸, —OSO₂R⁸, —SO₂NR⁸₂, —NR⁸SO₂R⁸, —OC(O)R⁸, —C(O)OR⁸, —C(O)NR⁸₂, —NR⁸C(O)R⁸, —OR⁸, or —NR⁸₂,

each R⁷ and R⁸ is, independently, hydrogen, lower alkyl, substituted lower alkyl, aryl, substituted aryl, aryl(lower)alkyl, substituted aryl(lower)alkyl, heteroaryl(lower)alkyl, substituted heteroaryl-(lower)alkyl, heterocyclyl, substituted heterocyclyl, heteroaryl, or substituted heteroaryl,

each Y is a non-interfering substituent,

each x is, independently, 0, 1 or 2, and

the urea linker connects a carbon which is designated c with a carbon which is designated d,

or a pharmaceutically acceptable salt thereof, in the form of a single stereoisomer or mixture of stereoisomers thereof.

In a first embodiment of this aspect of the invention, compounds of Formula I, or pharmaceutically acceptable salts thereof, in the pharmaceutical compositions are provided where no Y is linked to a naphthalene ring via an azo linkage and if R¹ and R² are both —SO₂OH, then (i) no Y is —SO₂OH; (ii) neither R⁵ nor R⁶ is —SO₂OR⁸ or —OSO₂R⁸; and (iii) R⁵ and R⁶ are not both selected from the group consisting of hydroxy and hydrogen unless at least one (Y)ₓ is (Y′)_{x′}, wherein x′ is 1 or 2 and Y′ is a halo radical.

In another aspect, this invention this invention includes a method of such treatment where the insulin receptor-activating compound is a compound of Formula II:

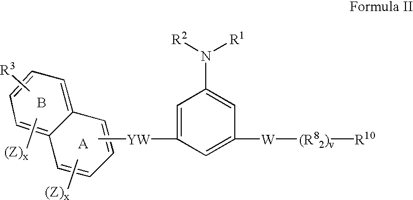

wherein:

R¹ and R² are, independently, hydrogen, lower alkyl, substituted lower alkyl, aryl, substituted aryl, —C(O)R⁴, —C(O)OR⁴, —C(O)NR⁴R⁵, —S(O)₂R⁴, —S(O)₂OR⁴, heteroaryl, substituted heteroaryl, heterocyclyl, substituted heterocyclyl, aryl(lower)alkyl, substituted aryl(lower)alkyl, heteroaryl(lower)alkyl, substituted heteroaryl-(lower)alkyl, or lower alkenyl, or R¹ and R² together with the conjoining nitrogen are C₃-C₉ heteroaryl, C₃-C₅ heterocyclyl, or —NO₂,

R³ is a substituent on the B ring and is —SO₂OR⁶, —C(O)OR⁶, —SO₂NR⁶₂, —C(O)NR⁶₂, or tetrazole;

each linker —WY— between the naphthyl and phenyl intersects the A ring on the naphthyl and is, independently, —C(O)NR⁷—, —NR⁷C(O)—, —C(O)O—, —OC(O)—, —CH═CH—, —NR⁷CH₂—, —CH₂NR⁷—, —NR⁷C(O)NR⁷—, —NR⁷C(O)O—, —OC(O)NR⁷—, —NR⁷SO₂O—, —OSO₂NR⁷—, —OC(O)O—, —SO₂NR⁷—, —NR⁷SO₂—, —OSO₂—, or —SO₂O—,

each R⁴ and R⁵ is, independently, hydrogen, lower alkyl, substituted lower alkyl, aryl, substituted aryl, aryl(lower)alkyl, substituted aryl(lower)alkyl, substituted heteroaryl, heteroaryl, heteroaryl-(lower)alkyl, substituted heteroaryl(lower)alkyl, heterocyclyl, substituted heterocyclyl, or lower alkenyl,

each R⁶ and R⁷ is, independently, hydrogen or lower alkyl,

each R⁸ is, independently, hydrogen, lower alkyl, substituted lower alkyl, aryl(lower)alkyl, substituted aryl(lower)alkyl, substituted heteroaryl, heteroaryl, heteroaryl(lower)alkyl, substituted heteroaryl(lower)alkyl, heterocyclyl, substituted heterocyclyl, lower alkenyl, nitro, halo, cyano, —OR⁹, —SR⁹, —C(O)R⁹, —OC(O)R⁹, —C(O)OR⁹, —NR⁹₂, —C(O)NR⁹₂, —NR⁹C(O)R⁹, —OSO₂R⁹, —SO₂OR⁹, —SO₂NR⁹₂, or —NR⁹SO₂R⁹,

each R⁹ is, independently, hydrogen, lower alkyl, substituted lower alkyl, aryl, substituted aryl, heteroaryl, substituted heteroaryl, heteroaryl-(lower)alkyl, substituted heteroaryl(lower)alkyl, heterocyclyl, substituted heterocyclyl, aryl(lower)alkyl, or substituted aryl(lower)alkyl,

each Z is a non-interfering substituent,

each x and v is, independently, 0, 1, 2 or 3, and

R¹⁰ is aryl, substituted aryl, heteroaryl, or substituted heteroaryl,

or a pharmaceutically acceptable salt thereof, in the form of a single stereoisomer or mixture of stereoisomers thereof.

In another aspect, this invention includes a method of such treatment where the insulin receptor-activating compound is a compound of Formula III:

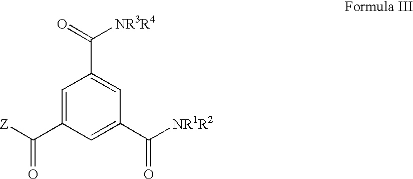

wherein:

R¹ and R² are, independently, hydrogen, lower alkyl, substituted lower alkyl, aryl, substituted aryl, heteroaryl, substituted heteroaryl, heterocyclyl, substituted heterocyclyl, aryl(lower)alkyl, substituted aryl(lower)alkyl, heteroaryl(lower)alkyl, substituted heteroaryl-(lower)alkyl, or lower alkenyl, or R¹ and R² together with the conjoining nitrogen are C₃-C₉ heteroaryl, or C₃-C₅ heterocyclyl.

Z is OH, halo, OR¹ or NR¹R² wherein R¹ and R² are as defined above,

or a pharmaceutically acceptable salt thereof, in the form of a single stereoisomer or mixture of stereoisomers thereof.

In another aspect, this invention includes a method of such treatment where the insulin receptor-activating compound is a compound of Formula IV:

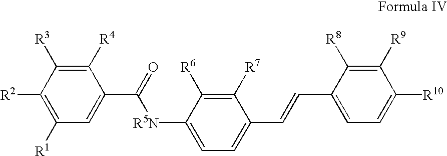

wherein

R¹, R³, and R⁴ are, independently, hydrogen, lower alkyl, substituted lower alkyl, halo, hydroxyl, substituted alkyloxy, carboxyl, —NR¹¹R¹², or —C(O)NR¹¹R¹²,

R² is hydrogen, lower alkyl, substituted alkyl, halo, hydroxyl, alkoxy, substituted alkyloxy, carboxyl, —NR¹¹R¹², —NR¹¹C(O)R¹², or —C(O)NR¹¹,

R⁵ is hydrogen, lower alkyl, substituted lower alkyl, or aryl,

R⁶and R⁷ are, independently, hydrogen or carboxyl,

R⁸and R⁹ are, independently, hydrogen, lower alkyl, substituted lower alkyl, halo, hydroxyl, alkoxy, carboxyl, —NR¹¹R¹², —C(O)NR¹¹R¹²

R¹⁰ is lower alkyl, substituted lower alkyl, halo, carboxyl, —C(O)NR¹¹R¹²

R¹¹ and R¹² are, independently, hydrogen, lower alkyl, substituted lower alkyl, aryl, substituted aryl, aryl(lower)alkyl, substituted aryl(lower)alkyl, heteroaryl-(lower)alkyl, -substituted heteroaryl(lower)alkyl, heterocyclyl, substituted heterocyclyl, heteroaryl, or substituted heteroaryl-C(O)-aryl, or aryl,

or a pharmaceutically acceptable salt thereof, in the form of a single stereoisomer or mixture of stereoisomers thereof.

In another aspect, this invention includes a method of such treatment where the insulin receptor-activating compound is a compound of Formula V:

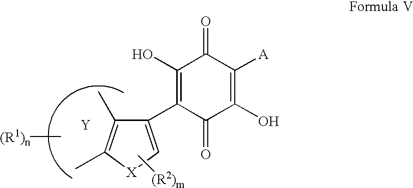

wherein

Ring Y represents a 5-6-membered aryl or heteroaryl fused ring, which is optionally substituted with 1-4 groups selected from R¹.

X represents O, S(O)m or N, wherein m is 0, 1 or 2;

A represents a member selected from the group consisting of:

(a) a 6-10-membered mono-or bicyclic aryl group

(b) a 5-6-membered isolated monocyclic heteroaryl group

(c) a 9-10-membered bicyclic heteroaryl group, attachmnent to which is through a 6-membered ring, or

(d) an 8-membered bicyclic heteroaryl group, the heteroaryl groups having 1-4 heteroatoms selected from O, S(O)ₘ and N, said aryl and heteroaryl groups being optionally substituted with 1-3 R¹ groups

R¹ is independently selected from:

halo, —OH, —C₁₋₁₂alkyl(R²)₃, —C₂₋₁₀alkenyl(R²)₃, —C₂₋₁₀alkynyl(R²)₃, —C₆₋₁₀aryl(R²)₃, -heteroaryl(R²)₃-heterocyclyl(R²)₃, —NH₂, —NHC₁₋₆alkyl(R²)₃, —N(C₁₋₆alkyl(R²)₃)₂, —N₃, —OC₁₋₆alkyl(R²)₃, —S(O)ₘHS(O)ₘC₁₋₆alkyl(R²)₃, —CHO, —C(O)C₁₋₆alkyl(R²)₃, —CO₂H, —C(O)OC₁₋₆ alkyl(R²)₃, —C(O)SC₁₋₆alkyl(R²)₃, —C(O)NH₂, C(O)NHC_{1-6,} alkyl(R²)₃, —NHC(O)₁₋₆alkyl(R²)₃, —S(O)ₘNH₂, —NHS(O)ₘC₁₋₆alkyl(R²)₃, —S(O)ₘNHC₁₋₆alkyl(R²)₃ and —S(O)ₘN(C₁₋₆alkyl(R²)₃)₂

wherein m is 0, 1 or 2;

R² is independently selected from:

H, OH, halo, —C₁₋₄ alkyl, —C₂₋₄ alkenyl, —C₂₋₄ alkynyl, —CF₃, —OCF₃, —NO₂, —N₃, —CHO, —OC₁₋₆alkyl, —S(O)ₘC₁₋₆alkyl, —NH₂, —NHC₁₋₆alkyl, —N(C₁₋₆ alkyl)₂, —C(O)C₁₋₆alkyl, —CO₂H, —CO₂C₁₋₆ alkyl, —C(O)NH₂, —C(O)NHC₁₋₆alkyl, —C(O)N(C₁₋₆alkyl)₂, —OC(O)C₁₋₆alkyl, —NHC(O)C₁₋₆ alkyl, —S(O)ₘNH₂, —S(O)ₘNHC₁₋₆alkyl, —S(O)ₘ(C₁₋₆alkyl)₂, aryl, heteroaryl and heterocyclyl,

wherein m is 0, 1 or 2,

or a pharmaceutically acceptable salt thereof, in the form of a single stereoisomer or mixture of stereoisomers thereof.

In another aspect, this invention includes a method of such treatment where the insulin receptor-activating compound is a compound of Formula VI:

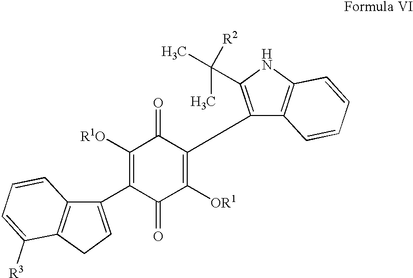

wherein

R¹ is hydrogen, or methyl,

R² is —CH₂CH₃ or —CH═CH₂,

R³ is —CH═CH—C(CH₃)═CH₂; CH₂—CH═C(CH₃)₂ or —CH₂—CH₂—CH═C(CH₃)₂,

or a pharmaceutically acceptable salt thereof, in the form of a single stereoisomer or mixture of stereoisomers thereof.

In another aspect, this invention includes a method of such treatment where the insulin receptor-activating compound is a compound of Formula VII:

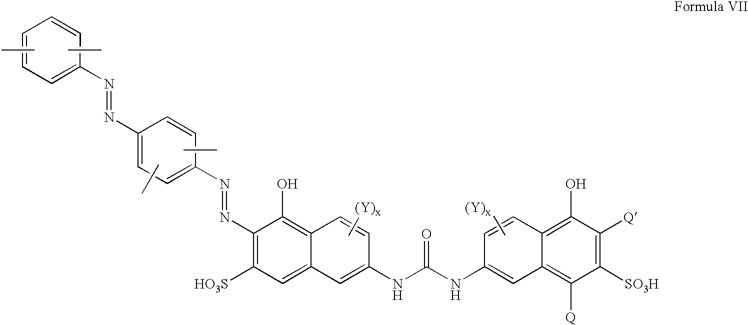

wherein

Q and Q′ are either hydrogen or

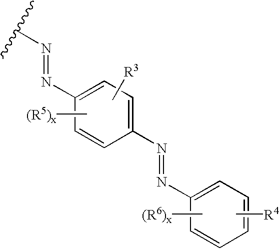

R¹ and R² are, independently, —SO₂NR⁷₂, —C(O)NR⁷₂, —NR⁷SO₂R⁷, —SO₂OR⁷, —C(O)OR⁷, —PO₃ R⁷₂, or tetrazolyl,

R³ and R⁴ are, independently, —SO₂NR⁷₂, —C(O)NR⁷₂, —NR⁷C(O)R⁷, —SO₂OR⁷, —C(O)OR⁷, —PO₃ R⁷₂, or tetrazolyl,

R⁵ and R⁶ are, independently, hydrogen, lower alkyl, substituted lower alkyl, cyano, halo, nitro, —SR⁸, —C(O)R⁸, —SO₂OR⁸, —OSO₂R⁸, —SO₂NR⁸₂, —NR⁸SO₂R⁸, —OC(O)R⁸, —C(O)OR⁸, —C(O)NR⁸₂, —NR⁸C(O)R⁸, —OR⁸, or —NR⁸₂,

each R⁷ and R⁸ is, independently, hydrogen, lower alkyl, substituted lower alkyl, aryl, substituted aryl, aryl(lower)alkyl, substituted aryl(lower)alkyl, heteroaryl(lower)alkyl, substituted heteroaryl-(lower)alkyl, heterocyclyl, substituted heterocyclyl, heteroaryl, or substituted heteroaryl,

each Y is a non-interfering substituent, and

each x is, independently, 0, 1 or 2,

or pharmaceutically acceptable salts thereof, optionally in the form of a single stereoisomer or mixture of stereoisomers thereof.

### BRIEF DESCRIPTION OF THE DRAWINGS

FIG. 1 shows the effect of indinavir and indinavir plus an insulin-receptor activating on blood glucose levels following oral glucose challenge.

FIG. 2 shows the effect of indinavir and indinavir plus an insulin receptor-activating compound on plasma insulin levels.

### DETAILED DESCRIPTION OF THE INVENTION

(a) Definitions:

“Alkyl”, as in “alkyl” or “alkyloxy”, means a C₁-C₂₀ monovalent hydrocarbyl moiety which may be linear, branched, or cyclic. “Lower alkyl”, as in “lower alkyl”, “halo-lower alkyl”, “aryl(lower)alkyl”, or “heteroaryl(lower)alkyl”, means a C₁-C₁₀ alkyl. The term “lower alkyl” includes such moieties as methyl, ethyl, isopropyl, propyl, butyl, isobutyl, sec-butyl, tert-butyl, pentyl, hexyl, cyclopentyl, cyclopropylmethyl, cyclohexyl, or cyclohexylmethyl. C₁-C₆ lower alkyls are preferred.

A substituted alkyl or substituted lower alkyl is an alkyl or lower alkyl, respectively, which is typically mono-, di-, or trisubstituted with a moiety such as aryl, substituted aryl, heteroaryl, nitro, cyano, halo, —OR, —SR, —C(O)R, —OC(O)R, —NRR′, —S(O)₂OR, —OS(O)₂R, —S(O)₂NRR′, —NRS(O)₂R′, —C(O)OR, —C(O)NRR′, or —NRC(O)R!, wherein R and R′ are, independently, hydrogen, lower alkyl, substituted lower alkyl, aryl, substituted aryl, heteroaryl, heteroaryl(lower)alkyl, substituted aryl(lower)alkyl, or aryl(lower)alkyl. Substituted alkyls or substituted lower alkyls which are substituted with one to three of the substituents selected from the group consisting of cyano, halo, lower alkyloxy, thio, nitro, amino, or hydroxy are particularly preferred.

The term “halo-lower alkyl” means a lower alkyl substituted with one to three halo groups, and is further exemplified by such radicals as —CF₃, —CH₂CF₃ and —CH₂CCl₃.

“Aryl”, as in “aryl”, “aryloxy”, and “aryl(lower)alkyl”, means a radical derived from an aromatic hydrocarbon containing 6 to 20 ring carbon atoms, having a single ring (e.g., phenyl), or two or more condensed rings, preferably 2 to 3 condensed rings (e.g., naphthyl), or two or more aromatic rings, preferably 2 to 3 aromatic rings, which are linked by a single bond (e.g., biphenyl). The aryl is preferably a C₆-C₁₆ aryl and even more preferably, a C₆-C₁₄ aryl.

A “substituted aryl” is an aryl radical which is substituted, multiply or singly, with a moiety such as an alkyl, substituted alkyl, halo, cyano, nitro, —OR, —SR, —C(O)R, —OC(O)R, —NRR′, —S(O)₂OR, —OS(O)₂R, —S(O)₂N′, —NRS(O)₂R′, —C(O)OR, —C(O)NRR′, or —NRC(O)R′, wherein R and R′ are, independently, hydrogen, lower alkyl, substituted lower alkyl, aryl, substituted aryl, heteroaryl, heteroaryl(lower)alkyl, aryl(lower)alkyl, or substituted aryl(lower)alkyl. A substituted aryl may be substituted from one to seven times with any combination of the radicals listed above. Preferably, however, the substituted aryl is mono-, di-, or trisubstituted. Especially preferred substituents on a substituted aryl are lower alkyl, halo-lower alkyl, halo, cyano, thio, nitro, amino, lower alkyloxy, or hydroxy. The radicals —S(O)₂OR, —S(O)₂NRR′, —C(O)OR, and —C(O)NRR′, wherein R and R′ are, independently, hydrogen or a lower alkyl, are also especially preferred substituents of substituted aryls on the compounds of the present invention.

“Heteroaryl”, as in heteroaryl and heteroaryl(lower)alkyl, means a radical derived from an aromatic hydrocarbon containing 5 to 14 ring atoms, 1 to 5 of which are heteroatoms chosen, independently, from N, O, or S, and includes monocyclic, condensed heterocyclic, and condensed carbocyclic and heterocyclic aromatic rings (e.g., thienyl, furyl, pyrrolyl, pyrimidinyl, isoxazolyl, oxazolyl, indolyl, isobenzofuranyl, purinyl, isoquinolyl, pteridinyl, imidazolyl, pyridyl, pyrazolyl, pyrazinyl, quinolyl, etc.).

A “substituted heteroaryl” may have from one to three substituents such as an alkyl, substituted alkyl, halo, cyano, nitro, —OR, —SR, —C(O)R, —OC(O)R, —NRR′, —S(O)₂OR, —OS(O)₂R, —S(O)₂NRR′, —NRS(O)₂R′, —C(O)OR, —C(O)NRR′, or —NRC(O)R′, wherein R and R′ are, independently, hydrogen, lower alkyl, substituted lower alkyl, aryl, substituted aryl, heteroaryl, heteroaryl(lower)alkyl, aryl(lower)alkyl, or substituted aryl(lower)alkyl. In addition, any two adjacent substituents on the heteroaryl may optionally together form a lower alkylenedioxy. Particularly preferred substituents on the substituted heteroaryl include hydroxy, halo, lower alkyloxy, cyano, thio, nitro, lower alkyl, halo-lower alkyl, halo-lower alkyl, or amino.

“Heterocyclyl” means a radical derived from an aliphatic, cyclic hydrocarbon containing 5 to 14 ring atoms, 1 to 5 of which are heteroatoms chosen, independently, from N, O, or S. Monocyclic rings (e.g., tetrahydrofuranyl, tetrahydropyranyl, piperidinyl, etc.) are preferred.

A “substituted heterocyclyl” may have from one to three substituents, preferably substituents such as an alkyl, substituted alkyl, halo, cyano, nitro, —OR, —SR, —C(O)R, —OC(O)R, —NRR′, —S(O)₂OR, —OS(O)₂R, —S(O)₂NRR′, —NRS(O)₂R′, ′C(O)OR, —C(O)NRR′, or —NRC(O)R′, wherein R and R′ are, independently, hydrogen, lower alkyl, substituted alkyl, aryl, substituted aryl, heteroaryl, heteroaryl(lower)alkyl, aryl(lower)alkyl, or substituted aryl(lower)alkyl. Preferred substituents on a substituted heterocyclyl include lower alkyl, halo-lower alkyl, halo, cyano, thio, amino, lower alkyloxy, or hydroxy.

“Aryl(lower)alkyl” means a lower alkyl radical which is substituted with an aryl, as previously defined. A “substituted aryl(lower)alkyl” means an aryl(lower)alkyl radical having one to three substituents on the aryl portion or the alkyl portion of the radical, or both.

“Heteroaryl(lower)alkyl” means a lower alkyl radical which is substituted with a heteroaryl, as previously defined. A “substituted heteroaryl(lower)aryl” means a heteroaryl(lower)alkyl radical having one to three substituents on the heteroaryl portion or the alkyl portion of the radical, or both.

A “lower alkyloxy” means an —OR radical, where R is a lower alkyl.

“Lower alkenyl” means any branched or unbranched unsaturated C₂-C₁₀ group having the number of carbon atoms specified, or up to 10 carbon atoms if no limitation on the number of carbon atoms is specified; and having 1 or more double bonds in the group. Lower alkenyl is exemplified by ethenyl, propenyl, butenyl, pentenyl, and hexenyl, in their various isomeric forms, where the unsaturated bond(s) can be located anywhere in the group.

“Halo” means bromo, iodo, fluoro, or chloro.

A “non-interfering substituent” means a substituent which, when present on a given compound, does not substantially decrease or otherwise inhibit a particular, desired bioactivity of the compound, such as the ability of the compound to reverse the glucose uptake inhibition caused by HIV protease-inhibitor drugs, to activate the insulin receptor, or to stimulate the uptake of glucose into cells. The presence of the non-interfering substituent should not detrimentally affect the bioactivity of the compound by more than about 30%. Preferably, the non-interfering substituent decreases the bioactivity of the compound by less than about 10%. Most preferably, the non-interfering substituent does not decrease the bioactivity of the compound to any detectable degree. However, the effect of the presence of the non-interfering substituent on the compound need not be neutral. For instance, the non-interfering substituent may optionally increase a particular bioactivity of the compound. Suitable non-interfering substituents include, but are not limited to, hydrogen, alkyl, substituted alkyl, cyano, halo, nitro, —SR, —OR, and —NRR′, wherein R and R′ are, independently, hydrogen, lower alkyl, or substituted lower alkyl.

A “pharmaceutically acceptable salt” may be any salt derived from an inorganic or organic acid or an inorganic or organic base. The term “pharmaceutically acceptable anion” refers to the anion of such acid addition salts, The term “pharmaceutically acceptable cation” refers to a cation formed by addition of a base. The salt and/or the anion or cation are chosen not to be biologically or otherwise undesirable,

“Stereoisomers” are compounds that have the same sequence of covalent bonds and differ in the relative disposition of their atoms in space.

“Inner salts” or “Zwitterions” can be formed by transferring a proton from the carboxyl group onto the lone pair of electrons of the nitrogen atom in the amino group.

“Therapeutically effective amount” means that amount which, when administered to an animal for treating a disease, is sufficient to effect such treatment for the disease.

“Treating” or “treatment” of a disease in a mammal includes:

(1) preventing the disease from occurring in a mammal which may be predisposed to the disease but does not yet experience or display symptoms of the disease,

(2) inhibiting the disease, i.e., arresting or slowing its development,

(3) relieving the disease, i.e., causing regression of the disease, or

(4) relieving the symptoms of the disease, i.e., lessening the effects of the disease.

“Disease” includes any unhealthy condition of a human, including, particularly, HIV protease inhibitor-induced insulin resistance, hyperglycemia, diabetes, ketoacidosis, lipodystrophy, and hypertriglyceridemia.

A metabolic disorder “induced by” treatment with an HIV protease inhibitor does not mean that the treatment is necessarily the sole cause of the disorder, but means merely that treatment with the protease inhibitor contributes to the disorder.

(b) Compounds of the Invention

In one aspect, this invention is directed to pharmaceutical compositions comprising (i) a pharmaceutically acceptable carrier and (ii) as an active ingredient, an insulin receptor-activating compound for the treatment of HIV protease inhibitor-induced insulin resistance, hyperglycemia, diabetes, lipodystrophy, hypertriglyceridemia, and ketoacidosis in humans. Such pharmaceutical compositions may comprise any insulin receptor-activating compound, including compounds of Formulas I to VII as herein disclosed.

Compounds useful in the practice of this invention may be prepared by methods familiar to one skilled in the art of chemistry. In addition, examples and syntheses of compounds of formula I-II are described in WO 00/71506 and WO 01/12591. Syntheses of compounds of Formulae III and IV are described generally below, and in U.S. patent application Ser. Nos. 09/872,763 (and Provisional Application No. 60/208,591) and 09/949,165 (and Provisional Application No. 60/230,738), respectively. Compounds of Formula V and VI (and their syntheses) are described in U.S. Pat. No. 6,051,597 and WO 99/51225. Compounds of Formula VII (and their syntheses) are described in U.S. Pat. Nos. 5,851,988 and 5,830,918. These documents are incorporated herein by reference.

Certain compounds of the invention may contain one or more chiral centers. In such cases, all stereoisomers also fall within the scope of this invention. The invention compounds include the individually isolated stereoisomers as well as mixtures of such stereoisomers.

The compounds of the invention further comprise pharmaceutically acceptable salts of the compounds disclosed herein. These pharmaceutically acceptable salts are suitable for use in all methods and pharmaceutical compositions of the present invention.

Pharmaceutically acceptable salts include salts which may be formed when acidic protons present are capable of reacting with inorganic or organic bases. Typically the parent compound is treated with an excess of an alkaline reagent, such as hydroxide, carbonate or alkoxide, containing an appropriate cation. Cations such as Na⁺, K⁺, Ca²⁺ and NH₄⁺ are examples of cations present in pharmaceutically acceptable salts. The Na⁺ salts are especially useful. Acceptable inorganic bases, therefore, include aluminum hydroxide, calcium hydroxide, potassium hydroxide, sodium carbonate and sodium hydroxide. Salts may also be prepared using organic bases, such as ethanolamine, diethanolamine, triethanolamine, N-methylglucamine, ethanolamine, and tromethamine.

If a compound of the invention contains a basic group, an acid addition salt may be prepared. Acid addition salts of the compounds are prepared in a standard manner in a suitable solvent from the parent compound and an excess of an acid, such as hydrochloric acid, hydrobromic acid, sulfuric acid (giving the sulfate and bisulfate salts), nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, salicylic acid, p-toluenesulfonic acid, hexanoic acid, heptanoic acid, cyclopentanepropionic acid, lactic acid, o-(4-hydroxy-benzoyl)benzoic acid, 1,2-ethanedisulfonic acid, 2-hydroxyethanesulfonic acid, benzenesulfonic acid, 4-chlorobenzenesulfonic acid, 2-naphthalenesulfonic acid, camphorsulfonic acid, 4-methyl-bicyclo[2.2.2.]oct-2-ene-1-carboxylic acid, glucoheptonic acid, gluconic acid, 4,4′-methylenebis(3-hydroxy-2-naphthoic)acid, 3-phenylpropionic acid, trimethylacetic acid, t-butylacetic acid, laurylsulfuric acid, glucuronic acid, glutamic acid, 3-hydroxy-2-naphthoic acid, stearic acid, muconic acid and the like.

Certain of the compounds of the invention may form inner salts or Zwitterions.

Pharmaceutical compositions of all the compounds of the present invention are contemplated. These pharmaceutical compositions comprise (i) an insulin receptor activator as an active ingredient and (ii) a pharmaceutically acceptable carrier. Further, these pharmaceutical compositions comprise (i) a compound of the invention as an active ingredient and (ii) a pharmaceutically acceptable carrier.

Pharmaceutical compositions of the compounds of this invention, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration, but may also be used for oral administration. It may be desirable to add excipients such as polyvinylpyrrolidinone, gelatin, hydroxycellulose, acacia, polyethylene glycol, mannitol, sodium chloride or sodium citrate. Alternatively, these compounds may be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax. The amount of solid carrier may vary, and is preferably between about 20 mg to about 1 g per dosage unit. The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation is preferably in the form of a syrup, elixir, emulsion or an aqueous or non-aqueous suspension. Such a liquid formulation may be administered directly p.o. or filled into a soft gelatin capsule.

(c) Methods of use of the Compounds of the Present Invention.

The insulin receptor-activating compounds of the present invention have been found to stimulate autophosphorylation of the insulin receptor. In addition, these compounds have been shown to enhance the transport of glucose into cultured fibroblast cells after the cells treatment with a variety of HIV protease inhibitors. Although applicants do not wish to be bound by theory, it is believed that HIV-protease inhibitors reduce the association of glucose transporters with the cell membrane, and that the compounds of the invention are effective to enhance glucose transport into HIV-protease inhibitor-affected cells by enhancing membrane-associative and other activity of glucose transporters.

The ability of the compounds of this invention to stimulate the uptake of glucose into cells indicates their usefulness in the treatment and management of patients who have HIV protease inhibitor-induced insulin resistance, hyperglycemia, diabetes, ketoacidosis, lipodystrophy, or hypertriglyceridemia. By virtue of the activities of the compounds of the invention, they may be used to stimulate the kinase activity of an insulin receptor, to enhance the activation of the insulin receptor by insulin, to enhance the stimulation by insulin of cellular glucose uptake, and to stimulate the uptake of glucose in subjects who have HIV protease therapy induced diabetes, ketoacidosis, insulin resistance, hyperglycemia, lipodystrophy, or hypertriglyceridemia. Thus, insulin receptor-activating compounds, and, more specifically, the compounds of the invention, may be used in the preparation of medicaments for the treatment of any such disease induced by the use of an HIV protease inhibitor.

The method may further comprise treating a patient who has complications due to HIV protease therapy with one or more additional forms of therapy for insulin resistance, hyperglycemia, diabetes, ketoacidosis, lipodystrophy, or hypertriglyceridemia, such as administering insulin to the patient. The insulin or other additional form of treatment may be delivered to the patient in an amount which is therapeutically effective when used in conjunction with a compound of the invention. This therapeutically effective amount of insulin or other additional form of treatment when used in combination with a compound of the invention may be less than the amount of insulin which would be therapeutically effective if delivered to the patient alone. It is understood that the insulin which is administered in any of the treatments of the present invention may either be isolated from a natural source or be recombinant insulin. In addition, an insulin analog may be substituted for insulin in any of the treatments of the present invention. Thus, a medicament prepared (for the treatment of any such disease induced by the use of an HIV protease inhibitor) utilizing an insulin receptor-activating compound, more specifically, a compound of the invention may further comprise an additional form of treatment, such as, for example, insulin or an insulin analog, of any such disease induced by the use of an HIV protease inhibitor.

The methods of the invention for treating HIV protease induced diabetes, ketoacidosis, lipodystrophy, hypertriglyceridemia, insulin resistance, or hyperglycemia by combination therapy may also comprise the administration of the compound of the invention to the patient in combination with a non-insulin, antidiabetic agent or other treatment for type II diabetes. For instance, the antidiabetic drug which is administered to the mammal in combination with a compound of the invention may optionally be a thiazolidinedione, such as troglitazone, or a sulphonylurea. The total amount of the combination of drugs (invention compound plus insulin, and/or other antidiabetic drug) administered to the mammal for the treatment of type 11 diabetes must be a therapeutically effective amount, although the amount of each of the individual drugs used in the combination therapy may be suboptimal for therapeutic purposes if that drug were to be delivered alone to the mammal with type II diabetes. Thus, a medicament prepared (for the treatment of any such disease induced by the use of an HIV protease inhibitor) utilizing an insulin receptor-activating compound, more specifically, a compound of the invention may further comprise an additional form of treatment, such as, for example, a non-insulin, antidiabetic agent, of any such disease induced by the use of an HIV protease inhibitor.

The methods of the invention for treating HIV protease induced diabetes, ketoacidosis, lipodystrophy, hypertriglyceridemia, insulin resistance, or hyperglycemia by combination therapy may also comprise the administration of a compound of the invention to the patient in combination with another compound of the invention. Thus, a medicament prepared (for the treatment of any such disease induced by the use of an HIV protease inhibitor) utilizing an insulin receptor-activating compound, more specifically, a compound of the invention may further comprise another compound of the invention.

The compounds of this invention can be used in the preparation of a medicament for the treatment of a disease induced by the use of an HIV protease inhibitor.

The compounds of this invention are thus used to enhance glucose uptake in patients on HIV protease therapy which require such treatment. The method of treatment comprises the administration of an effective quantity of the chosen compound of the invention, preferably dispersed in a pharmaceutical carrier. Preferred routes of administration are the parenteral and oral routes.

The invention compounds may be administered by any route suitable to the subject being treated and the nature of the subject's disease. Routes of administration include, but are not limited to, administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in Remington's Pharmaceutical Sciences, 20th edition, Mack Publishing Company, Easton, Pa.

Dosage units of the active ingredient are generally selected from the range of 0.01 to 1000 mg/kg, preferably 0.01 to 100 mg/kg and more preferably 0.1 to 50 mg/kg, but will be readily determined by one skilled in the art depending upon the route of administration, age and disease of the patient. The compounds of the invention are most preferably administered in a dosage unit of 1 to 10 mg/kg. These dosage units may be administered one to ten times daily for acute or chronic disease.

### EXAMPLES

The Examples which follow serve to illustrate this invention, and are not intended to limit the scope of this invention, but are provided to show how to make and use the compounds of this invention.

The compounds of this invention are prepared by conventional methods of organic chemistry. In some cases, protective groups may be introduced and finally removed. Suitable protective groups for amino, hydroxy, carboxyl groups are described in Greene, et al., Protective Groups in Organic Synthesis, Second Edition, John Wiley and Sons, New York, 1991. Activation of carboxylic acids can be achieved by using a number of different reagents as described in Larock, Comprehensive Organic Transformations, VCH Publishers, New York, 1989.

The compounds of Formula III are prepared by conventional methods of organic chemistry.

Generally, a compound of the formula

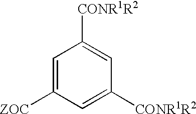

wherein:

R¹ and R² are, independently, hydrogen, lower alkyl, substituted lower alkyl, aryl, substituted aryl, heteroaryl, substituted heteroaryl, heterocyclyl, substituted heterocyclyl, aryl(lower)alkyl, substituted aryl(lower)alkyl, heteroaryl(lower)alkyl, substituted heteroaryl(lower)alkyl, or lower alkenyl, or

R¹ and R² together with the conjoining nitrogen are C₃-C₉ heteroaryl, or C₃-C₅ heterocyclyl, and

Z is OH, Cl, Br, F, OR¹ or NR¹R² wherein R¹ and R² are as defined above, or a pharmaceutically acceptable salt thereof, or a single stereoisomer or mixture of stereoisomers thereof;

may be prepared by a process comprising:

a) reaction of a 1,3,5-benzenetricarbonyl trihalide, wherein the halide is selected from the group consisting of Cl, Br, and F, with at least 1 to at least 3 moles of HNR¹R² wherein R¹ and R² are as defined above; or

(b) reaction of an activated carboxy di-amide of the formula

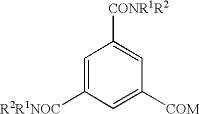

with a primary amine R³NH₂ or a secondary amine R³R⁴NH, wherein M is any substituent that will allow reaction with said amine; for example, M can be halogen, formate, imidazole, or some other promoting or coupling reagent; or

(c) acid esterification of a compound of the formula

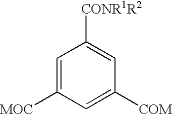

to form a compound of formula A; or

(d) chemical elaboration of one or more substituents of substituted R¹ or R², wherein said substituent is convertible into another substituent; or

(e) conversion of a compound of Formula A with three NR¹R² groups to a compound of Formula I with one or two NR¹R² groups, or

(f) conversion of the compound of Formula A to a pharmaceutically acceptable salt; or

(g) conversion of a salt of the compound of Formula A to a free compound; or

(h) conversion of a salt of the compound of Formula A to a pharmaceutically acceptable salt; or

(i) resolution of a racemic mixture of any proportions of the compound of Formula A to yield a stereoisomer thereof.

Asymmetric compounds of Formula III can be prepared via the following reaction scheme:

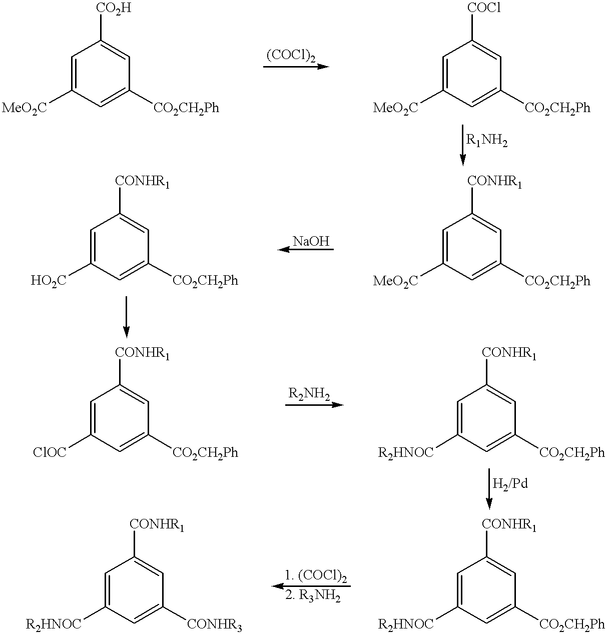

A benzyl ester of the compound of the invention is hydrolyzed (under basic hydrolysis conditions or by catalytic hydrogenolysis) to yield a carboxylic acid of the compound; this carboxylic acid compound is subsequently converted into an acid cloride of the compound, which is reacted with an amine to yield an asymmetric form of the compound of the invention.

As shown above, a differentially protected, mono-methyl, mono-benzyl esters of benzene tricarboxylic acid may be converted to the acid chloride using oxalyl chloride to give the corresponding mono-acylchloride. Reaction with an amine affords a mono-amido compound Selective saponification of the methyl ester under basic conditions, followed by an acidic workup will give another carboxylic acid which may be converted to the acid chloride using oxalyl chlorides Reaction with a second amine will now give an unsymmetrical compound. The final carboxylic ester may be converted to the carboxylic acid by more forcing saponification conditions, or by catalytic hydrogenolysis. After isolation of the carboxylic acid, one can covert the carboxylic acid into the acid chloride and then react with a third amine to give benzene compound with three differing amido substituents. It will be evident to a person of ordinary skill in the art that diamines may be used in place of monoamines in the above synthesis.

In some cases, protective groups may be introduced and later removed. Suitable protective groups for amino, hydroxyl, carboxyl groups are described in Greene, et al., Protective Groups in Organic Synthesis, Second Edition, John Wiley and Sons, New York, 1991. Activation of carboxylic acids can be achieved by using a number of different reagents as described in Larock, Comprehensive Organic Transformations, VCH Publishers, New York, 1989.

The compounds of Formula IV are prepared by conventional methods of organic chemistry.

Generally, the process for preparation comprises:

(a) reaction of an iodo bis-amide compound of the formula

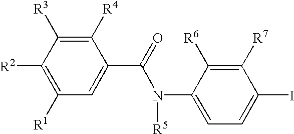

wherein R¹-R⁷ are as defined above, with a styrene or substituted styrene of the formula

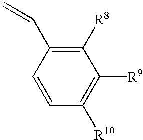

wherein R⁸-R¹⁰ are as defined above; or

(b) chemical elaboration of one or more substituents R¹-R¹⁰ wherein said substituent is convertible into another substituent R¹-R¹⁰; or

(c) introduction of a substituent R¹-R¹⁰ into one, two or all three of the phenyl rings; or

(d) deprotection of a protected group; or

(e) salt formation or interconversion; or

(f) ester hydrolysis; or

(g) liberation of a free acid or base of a compound of Formula IV, wherein R¹-R¹² are as defined above; or

(h) stereoisomer separation or synthesis.

The reaction of the iodo bis-amide compound with the styrene or substituted styrene shown in (a), above, can be carried out between 40° C. and 120° C. in the presence of such solvents as DMF, toluene, methylene chloride, or the like.

Chemical elaboration of one or more substituents R¹-R¹⁰ via the conversion of one such substituent into another substituent may be accomplished via hydrolysis, salt formation, acidification, alkylation, esterification, oxidation, or reduction.

In hydrolysis, an ester or amide compound is dissociated by reaction with water. Hydrolysis is catalyzed by acid or base, and hydrolysis of an amide generally requires more vigorous conditions (for example, a higher concentration of sulfuric acid at 1° C. to 100° C. for 1 to 5 hours) than those required for the hydrolysis of esters. Hydrolysis reactions can also be carried out with aqueous hydrochloric acid at 100° C. to 150° C. and may require as long as 18 hours.

In salt formation, a free acid is converted into a salt via addition of a basic reagent, such as aqueous sodium hydroxide or triethanolamine, that replaces all or part of the hydrogen ions of the acid with one or more cations of a base. The conversion of a compound into its corresponding acid addition salt is accomplished via treatment with a stoichiometric amount of an appropriate acid, such as hydrochloric acid. Typically, the free base is dissolved in a polar organic solvent, such as methanol or ethanol, and the acid is added in methanol or ethanol. The temperature is maintained at 0° C. to 50° C. The corresponding salt precipitates spontaneously or can be brought out of solution with a less polar solvent. In acidification, a chemical compound is converted into an acid.

In alkylation, an alkyl group is added to or substituted in a compound. Alkylation is carried out in a suitable solvent, such as acetonitrile, DMF, or THF, at 0° C. to 160° C., typically at approximately 25° C. to reflux, and requires some 1 to 18 hours.

An esterification reaction results in the formation of at least one ester product. In brief, the compound is reacted with from 1.0 to 5.0, preferable 2.0, molar equivalents of an alkanol, a thiol or ammonia, a monoalkyl, or dialkylamine, or a heterocyclic aminoalkanol, optionally in the presence of from 1.0 to 1.5, preferably 1.25, molar equivalents of a tertiary organic base such as 4-dimethylaminopyridine or, preferably, triethylamine, in an organic solvent such as dioxane, tetrahydroflran, or, preferably, dichloromethane. The reaction takes place at −10° C. to 50° C., preferably at ambient temperature, for 1 to 24 hours, preferably 4 hours.

Examples of syntheses of specific compounds usable in the invention follow.

### Example 1

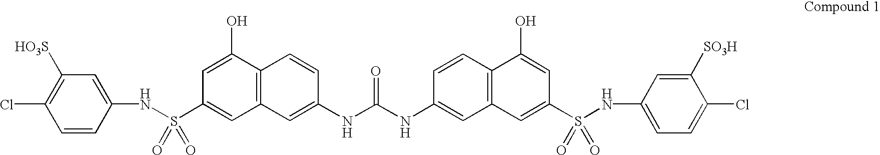

Preparation of Compound 1

4-hydroxy-7-{[(5-hydroxy-7-sulfo(2-naphthyl))amino]carbonylamino}naphthalene-2-sulfonic acid disodium salt.

To 10.77 g (0.045 moles) of 7-amino-4-hydroxynaphthalene-2-sulfonic acid dissolved in 45 mL of 1 N aqueous NaOH and 50 mL of water was added 3.70 g (0.045 moles) of sodium acetate. The pH of the solution was above 9. The reaction was cooled to under 5° C. in an ice-water bath. Then, 2.23 g (0.045 mole) of triphosgene dissolved in 15 mL of THF was added in three portions. The pH of the reaction fell to 4-5 and was readjusted to 7-8 by the dropwise addition of 1N aqueous NaOH. TLC (6:2:1 ethyl acetate:isopropanol:water) indicated the reaction was incomplete. Another 2.20 grams (0.045 moles) of triphosgene in 10 mL of THF was added portionwise with the pH kept above 7 by the addition of 1N aqueous NaOH. When the reaction was judged complete by TLC, the pH was lowered to 1 with aqueous HCl and the volatiles were removed by rotary evaporation. The solid product was collected by vacuum filtration. This resulted in the recovery of 10.85 g of the desired compound.

7-{[(7-(chlorosulfonyl)-5-hydroxy(2-naphthyl))amino]carbonylamino}-4-hydroxynaphthalene-2-sulfonyl chloride.

To 500 mg (0.912 mmol) of 4-hydroxy-7-{[(5-hydroxy-7-sulfo(2-naphthyl))amino]carbonylamino} naphthalene-2-sulfonic acid disodium salt suspended in 8 mL of phosphorous oxychloride was added 25 mL of 1:1 (v:v) sulfolane:acetonitrile and 0.5 mL of dimethylacetamide. The reaction mixture was allowed to stir at ambient temperature for 16 hours. The reaction became a clear solution which was poured onto 500 mL of ice. The ice mixture was placed in an ice bath and allowed to warm to room temperature. The resulting solid was collected by vacuum filtration and was washed with water. The solid was dried under high vacuum for 24 hours. This provided 412 mg of desired compound.

5-{[(7-{[N-(7-{[(4-chloro-3-benzenesulfonic acid)amino]sulfonyl}-5-hydroxy-(2-naphthyl)carbamoyl]amino}-4-hydroxy(2-naphthyl))sulfonyl]amino)-2-chlorobenzensulfonic acid.

To 0.15 g (0.277 mmol) of 7-{[(7-(chlorosulfonyl)-5-hydroxy-(2-naphthyl))amino]carbonylamino}-4-hydroxynaphthalene-2-sulfonyl chloride was added 1.5 mL of freshly distilled THF followed by 0.105 g (0.610 mmol) of 5-amino-2-chloro-benzenesulfonic acid. To this solution was added 67 uL (0.831 mmol) of pyridine. The reaction was allowed to stir at ambient temperature for 16 hours. Then, the reaction was partitioned between 1N HCl (aqueous) and ethyl acetate. The aqueous layer was extracted a second time with ethyl acetate and the combined organic layers were dried (MgSO4), filtered and volatiles removed by rotary evaporation. This provided 0.14 g of the desired compound.

### Example 2

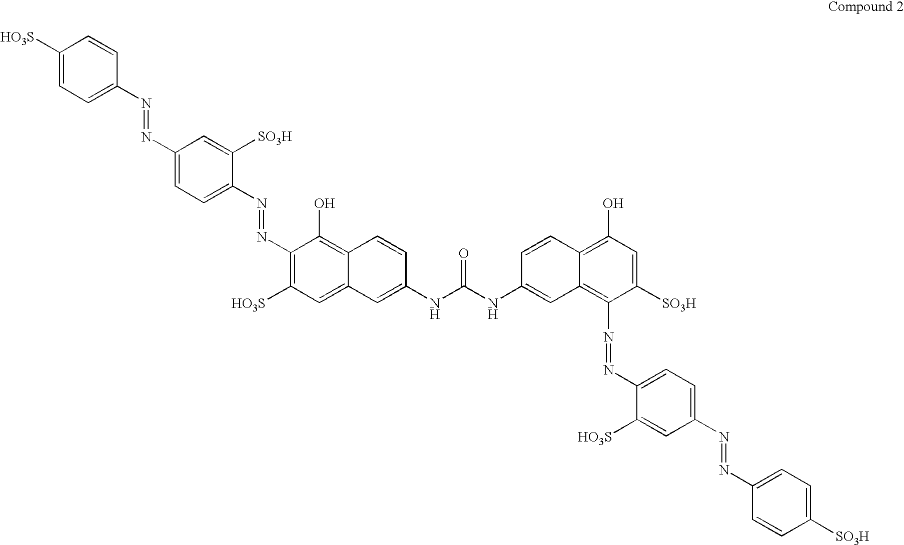

Compound 2 was prepared as described in U.S. Pat. No. 5,851,988.

### Example 3

Reversal of HIV Protease Inhibitor Block of Glucose Transport in Cells.

Stimulation of the insulin receptor leads to the transport of glucose from the blood into cells, thus modulating blood glucose levels. 3T3 L1 fibroblasts (ATCC) were grown in Dulbecco's modified Eagle's medium (DMEM) with 10% fetal bovine serum (FBS). The cells were plated at a density of 3×10⁴ cells/ml in 96 well plates (0.1 ml/well). Two days after confluence was reached, the cells were treated for 3 days with 0.5 mM isobutylmethylxanthine (IBMX), 1 μM dexamethasone, and 1.7 μM insulin. The cells were then transferred to DMEM with 10% FBS and supplemented with 1.7 μM insulin for 2 more days. The cells were maintained in DMEM with 10% FBS for an additional 4 days. Finally, the cells were serum starved overnight in 0.1% bovine serum albumin (BSA) in DMEM.

The following day, the cells were pretreated with Indinavir, Ritonavir, or Amprenavir (at the concentrations indicated in the Tables below) in a buffer containing 150 mM NaCl, 1.7 mM KCl, 0.9 mM CaCl₂ 1.47 mM K₂HPO₄ (pH 7.4) and 0.01% BSA for 6 minutes followed by treatment with a compound described in this patent (56 μM compound 1; 20 μM compound 2) with or without 100 nM insulin for 30 minutes. Following incubation, the cells were labeled with ¹⁴C-labeled 2-deoxy-D-glucose (0.5 μCi/ml) and incubation was continued for additional 30 minutes at 37° C. The cells were then washed 3 times with ice-cold PBS/20 mM glucose and lysed in 100 μl of lysis buffer (50 mM Hepes pH 7.6, 1% Triton X-100) for 30 minutes at room temperature. Radioactivity in the lysate was quantified by scintillation counting.

Once ¹⁴C-2-deoxy-D-glucose is transported into the cells, it is not released. Glucose transport is, therefore, proportional to the amount of radioactivity in the lysate.

Results:

Compounds 1 and 2 were demonstrated to increase the glucose transport activity in 3T3 L1 adipocytes which was otherwise inhibited by presence of commercial HIV protease inhibitor drugs, Ritonavir, Indinavir, or Amprenavir. Each of these drugs is known to induce insulin-resistance and other related disturbances in metabolism, such as lipodystrophy and hypertriglyceridemia, in patients treated with these HIV protease inhibitors.

The results are summarized below:

Indinavir

| Compound | 5 μM Indinavir | 10 μM Indinavir | 20 μM Indinavir |
|---|---|---|---|
| 1 | 1.34 fold over | 1.16 fold over | 1.12 fold over |
| | 50 nM insulin | 50 nM insulin | 50 nM insulin |
| 2 | 1.38 fold over | 1.25 fold over | 1.02 fold over |
| | 50 riM insulin | 50 nM insulin | 50 nM insulin |

Amprenavir

| | 5 μM | | |
|---|---|---|---|
| Compound | Amprenavir | 10 μM Amprenavir | 20 μM Amprenavir |
| 1 | 1.83 fold over | 1.53 fold over | 1.21 fold over |
| | 50 nM insulin | 50 nM insulin | 50 nM insulin |
| 2 | 1.25 foldover | 1.07 foldover | — |
| | 50 nM insulin | 50 nM insulin | |

Ritonavir

| Compound | 5 μM Ritonavir | 10 μM Ritonavir | 20 μM Ritonavir |
|---|---|---|---|
| 1 | 1.32 foldover | — | — |
| | 50 nM insulin | | |

### Example 4

[chemical structure]

Preparation of Compound 3

Compound 3 was synthesized by a method similar to the method disclosed in Example 1, except that the final steps following synthesis of 7-{[(7-(chlorosulfonyl)-5-hydroxy-(2-naphthyl))amino]carbonylamino}-4-hydroxynaphthalene-2-sulfonyl chloride were as described below:

5-{[(7-{[N-(7-{[(3-carboxy-4-chlorophenyl)amino]sulfonyl}-5-hydroxy-(2-naphthyl))carbamoyl]amino}-4-hydroxy(2-naphthyl))sulonyl]amino)-2-chlorobenzoic acid.

To 0.15 g (0.277 mmol) of 7-{[(7-(chlorosulfonyl)-5-hydroxy-(2-naphthyl))amino]-carbonylamino}-4-hydroxynaphthalene-2-sulfonyl chloride was added 1.5 mL of freshly distilled THF followed by 0.105 g (0.610 mmol) of 5-amino-2-chlorobenzoic acid. To this solution was added 67 uL (0.831 mmol) of pyridine. The reaction was allowed to stir at ambient temperature for 16 hours. Then, the reaction was partitioned between 1N HCl (aqueous) and ethyl acetate. The aqueous layer was extracted a second time with ethyl acetate and the combined organic layers were dried (MgSO4), filtered and volatiles removed by rotary evaporation. This provided 0.14 g of the desired compound.

### Example 5

Reversal of Protease Inhibitor-mediated Insulin Resistance in Normal Rats by Compound 1

Seven to nine week-old male CD rats (Charles River Laboratories, Hollister, Calif.) were used to study the effects of protease inhibitor and compound 1. Animals were kept in a 12 h/12 h light/dark cycle and were fasted overnight.

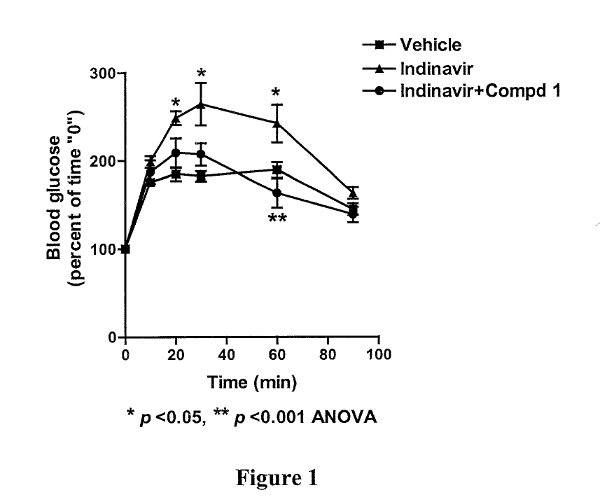

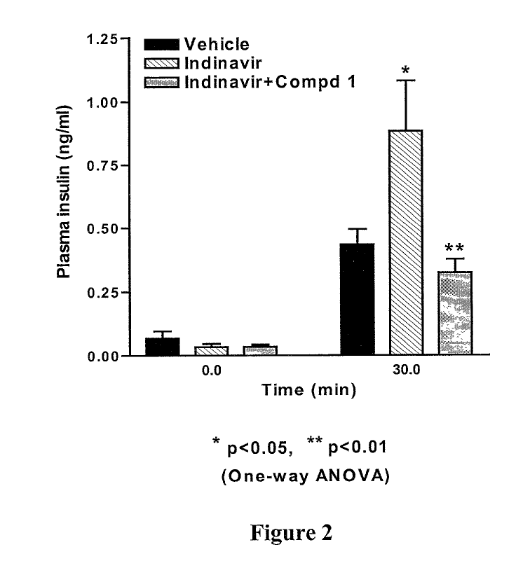

Indinavir sulfate was prepared in water, and compound 1 was prepared in PBS. Seven animals (average weight 300 grams) were used in each treatment condition. The animals were treated with vehicle or indinavir sulfate or indinavir sulfate plus compound 1 by oral administration. After 30 min, the animals were challenged with oral glucose load (2 g/kg), and blood glucose measurements were taken at 0 min, 10 min, 20 min, 30 min, 60 min, 90 min and 120 min by tail bleeding. Glucose measurements were made with a Glucometer and Glucose strips (Bayer). The resulting data are shown in FIGS. 1 and 2.

FIG. 1 shows the effect of indinavir and indinavir plus an insulin-receptor activating on blood glucose levels following oral glucose challenge. The blood glucose levels are reported as the percentage of the values before challenge (“zero time” values).

FIG. 2 shows the effect of indinavir and indinavir plus an insulin receptor-activating compound on plasma insulin levels. Blood samples obtained from 0 min and 30 min time points were analysed for plasma insulin levels by ELISA (ALPCO Diagnostics, Windham, N.H.).

Both Figures show the benefit of compounds of this invention.

We claim:

1. A method of treating a metabolic disorder in a person induced by treatment of the person with an HIV protease inhibitor, comprising administering to the person a therapeutically effective dose of an insulin receptor-activating compound that is not insulin, or a pharmaceutically acceptable salt thereof.

2. The method of claim 1, where the metabolic disorder induced by treatment with an HIV protease inhibitor is selected from the group consisting of insulin resistance, hyperglycemia, diabetes, ketoacidosis, lipodystrophy, and hypertriglyceridemia.

3. The method of claim 1, where the insulin receptor-activating compound is a compound of Formula I [chemical structure]
wherein:
R¹ and R² are substituents on the A ring and are, independently, —SO₂NR⁷₂, —C(O)NR⁷₂, —NR⁷SO₂R⁷, —NR⁷C(O)R⁷, —SO₂OR⁷, —C(O)OR⁷, —OSO₂R⁷, or —OC(O)R⁷,
R³ and R⁴ are, independently, hydrogen or lower alkyl, or R³ and R⁴ together are —(CH₂)₂—, —(CH₂)₃—, or —(CH₂)₄—,
R⁵ and R⁶ are, independently, hydrogen, lower alkyl, substituted lower alkyl, cyano, halo, nitro, —SR⁸, —C(O)R⁸, —SO₂OR⁸, —OSO₂R⁸, —SO₂NR⁸₂, —NR⁸SO₂R⁸, —OC(O)R⁸, —C(O)OR⁸, —C(O)NR⁸₂, —NR⁸C(O)R, —OR⁸, or —NR⁸₂,
each R⁷ and R⁸ is, independently, hydrogen, lower alkyl, substituted lower alkyl, aryl, substituted aryl, aryl(lower)alkyl, substituted aryl(lower)alkyl, heteroaryl(lower)alkyl, substituted heteroaryl-(lower)alkyl, heterocyclyl, substituted heterocyclyl, heteroaryl, or substituted heteroaryl,
each Y is a non-interfering substituent,
each x is, independently, 0, 1 or 2, and
the urea linker connects a carbon which is designated c with a carbon which is designated d,
or a pharmaceutically acceptable salt thereof, optionally in the form of a single stereoisomer or mixture of stereoisomers thereof.

4. The method of claim 3, wherein no Y is linked to a naphthalene ring via an azo linkage in the compound.

5. The method of claim 3, wherein, where R¹ and R² are both —SO₂OH,
(i) no Y is —SO₂OH;
(ii) neither R⁵ nor R⁶ is —SO₂OR⁸ or —OSO₂R⁸; and
(iii) where no (Y)ₓ is (Y′)_{x′}, wherein x′ is 1 or 2 and Y′ is a halo radical, R⁵ and R⁶ are not both selected from the group consisting of hydroxy and hydrogen.

6. The method of claim 1, where the insulin receptor-activating compound is a compound of Formula II [chemical structure]
wherein:
R¹ and R² are, independently, hydrogen, lower alkyl, substituted lower alkyl, aryl, substituted aryl, —C(O)R⁴, —C(O)OR⁴, —C(O)NR⁴R⁵, —S(O)₂R⁴, —S(O)₂OR⁴, heteroaryl, substituted heteroaryl, heterocyclyl, substituted heterocyclyl, aryl(lower)alkyl, substituted aryl(lower)-alkyl, heteroaryl(lower)alkyl, substituted heteroaryl(lower)alkyl, or lower alkenyl, or R¹ and R² together with the conjoining nitrogen are C₃-C₉ heteroaryl, C₃-C₅ heterocyclyl, or —NO₂,
R³ is a substituent on the B ring and is —SO₂OR , —C(O)OR⁸, —SO₂NR⁶₂, —C(O)NR⁶₂, or tetrazole;
each linker —WY— between the naphthyl and phenyl intersects the A ring on the naphthyl and is, independently, —C(O)NR⁷—, —NR⁷C(O)—, —C(O)O—, —OC(O)—, —CH═CH—, —NR⁷CH₂—, —CH₂NR⁷—, —NR⁷C(O)NR⁷—, —NR⁷C(O)O—, —OC(O)NR⁷—, —NR⁷SO₂O—, —OSO₂NR⁷—, —OC(O)O—, —SO₂NR⁷—, —NR⁷SO₂—, —OSO₂—, or —SO₂O—,
each R⁴ and R⁵ is, independently, hydrogen, lower alkyl, substituted lower alkyl, aryl, substituted aryl, aryl(lower)alkyl, substituted aryl(lower)alkyl, substituted heteroaryl, heteroaryl, heteroaryl-(lower)alkyl, substituted heteroaryl(lower)alkyl, heterocyclyl, substituted heterocyclyl, or lower alkenyl,
each R⁶ and R⁷ is, independently, hydrogen or lower alkyl,
each R⁸ is, independently, hydrogen, lower alkyl, substituted lower alkyl, aryl(lower)alkyl, substituted aryl(lower)alkyl, substituted heteroaryl, heteroaryl, heteroaryl(lower)alkyl, substituted heteroaryl(lower)alkyl, heterocyclyl, substituted heterocyclyl, lower alkenyl, nitro, halo, cyano, —OR⁹, —SR⁹, —C(O)R⁹, —OC(O)R⁹, —C(O)OR⁹, —NR⁹₂, —C(O)NR⁹₂, —NR⁹C(O)R⁹, —OSO₂R⁹, —SO₂OR⁹, —SO₂NR⁹₂, or —NR⁹SO₂R⁹,
each R⁹ is, independently, hydrogen, lower alkyl, substituted lower alkyl, aryl, substituted aryl, heteroaryl, substituted heteroaryl, heteroaryl-(lower)alkyl, substituted heteroaryl(lower)alkyl, heterocyclyl, substituted heterocyclyl, aryl(lower)alkyl, or substituted aryl(lower)alkyl,
each Z is a non-interfering substituent,
each x and v is, independently, 0, 1, 2 or 3, and
R¹⁰ is aryl, substituted aryl, heteroaryl, or substituted heteroaryl,
or a pharmaceutically acceptable salt thereof, optionally in the form of a single stereoisomer or mixture of stereoisomers thereof.

7. The method of claim 1, where the insulin receptor-activating compound is a compound of Formula III 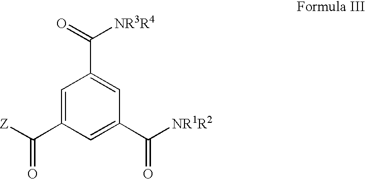
wherein:
R¹ and R² are, independently, hydrogen, lower alkyl, substituted lower alkyl, aryl, substituted aryl, heteroaryl, substituted heteroaryl, heterocyclyl, substituted heterocyclyl, aryl(lower)alkyl, substituted aryl(lower)-alkyl, heteroaryl(lower)alkyl, substituted heteroaryl(lower)alkyl, or lower alkenyl, or R¹ and R² together with the conjoining nitrogen are C₃-C₉ heteroaryl, or C₃-C₅ heterocyclyl,
Z is OH, halo, OR¹ or NR¹R²,
or a pharmaceutically acceptable salt thereof, optionally in the form of a single stereoisomer or mixture of stereoisomers thereof.

8. The method of claim 1, where the insulin receptor-activating compound is a compound of Formula IV [chemical structure]
wherein
R¹, R³, and R⁴ are, independently, hydrogen, lower alkyl, substituted lower alkyl, halo, hydroxyl, substituted alkyloxy, carboxyl, —NR¹¹R¹², or —C(O)NR¹¹R¹²,
R² is hydrogen, lower alkyl, substituted alkyl, halo, hydroxyl, alkoxy, substituted alkyloxy, carboxyl, —NR¹¹R¹², —NR¹¹C(O)R¹², or —C(O)NR¹¹,
R⁵ is hydrogen, lower alkyl, substituted lower alkyl, or aryl,
R⁶ and R⁷ are, independently, hydrogen or carboxyl,
R⁸ and R⁹ are, independently, hydrogen, lower alkyl, substituted lower alkyl, halo, hydroxyl, alkoxy, carboxyl, —NR¹¹R¹², or —C(O)NR¹¹R¹²,
R¹⁰ is lower alkyl, substituted lower alkyl, halo, carboxyl, —C(O)NR¹¹R¹²,
R¹¹ and R¹² are, independently, hydrogen, lower alkyl, substituted lower alkyl, aryl, substituted aryl, aryl(lower)alkyl, substituted aryl(lower)alkyl, heteroaryl-(lower)alkyl, -substituted heteroaryl(lower)alkyl, heterocyclyl, substituted heterocyclyl, heteroaryl, or substituted heteroaryl-C(O)-aryl, or aryl,
or a pharmaceutically acceptable salt thereof, optionally in the form of a single stereoisomer or mixture of stereoisomers thereof.

9. The method of claim 1, where the insulin receptor-activating compound is a compound of Formula V 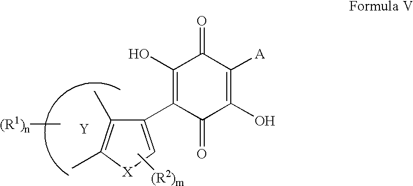
wherein
Ring Y represents a 5-6-membered aryl or heteroaryl fused ring, which is optionally substituted with 1-4 groups selected from R;
X represents O, S(O)m or N, wherein m is 0, 1 or 2;
A represents a member selected from the group consisting of:
(a) a 6-10-membered mono-or bicyclic aryl group,
(b) a 5-6-membered isolated monocyclic heteroaryl group,
(c) a 9-10-membered bicyclic heteroaryl group, attachment to which is through a 6-membered ring, or
(d) an 8-membered bicyclic heteroaryl group, the heteroaryl groups having 1-4 heteroatoms selected from O, S(O)m and N, said aryl and heteroaryl groups being optionally substituted with 1-3 R¹ groups;
R¹ is independently selected from:
halo, —OH, —C₁₋₁₂alkyl(R²)₃, —C₂₋₁₀alkenyl(R²)₃, —C₂₋₁₀alkynyl(R²)₃, —C₆₋₁₀aryl(R²)₃, —heteroaryl(R²)₃, —heterocyclyl(R²)₃, —NH₂, —NHC₁₋₆alkyl(R²)₃, —N(C₁₋₆alkyl(R²)₃, —N₃, —OC₁₋₆alkyl(R²)₃, —S(O)ₘH, S(O)ₘC₁₋₆alkyl(R²)₃, —CHO, —C(O)C₁₋₆alkyl(R²)₃, —CO₂H, —C(O)OC₁₋₆ alkyl(R²)₃, —C(O)SC₁₋₆alkyl(R²)₃, —C(O)NH₂, —C(O)NHC₁₋₆, alkyl(R²)₃, —NHC(O)C₁₋₆alkyl(R²)₃, —S(O)ₘNH₂, NHS(O)ₘC₁₋₆alkyl(R²)₃, —S(O)ₘNHC₁₋₆alkyl(R²)₃ and —S(O)ₘN(C₁₋₆alkyl(R²)₃)₂ wherein m is 0, 1, or 2;
R is independently selected from:
H, OH, halo, —C₁₋₄ alkyl, —C₂₋₄ alkenyl, —C₂₋₄ alkynyl, —CF₃, —OCF₃, —NO₂, —N₃, —CHO, —OC₁₋₆alkyl, —S(O)ₘCl₁₋₆alkyl, —NH₂, —NHC₁₋₆alkyl, —N(C₁₋₆alkyl)₂, —C(O)C₁₋₆alkyl, —CO₂H, —CO₂C₁₋₆alkyl, —C(O)NH₂, —C(O)NHC₁₋₆alkyl, —C(O)N(C₁₋₆alkyl)₂, —OC(O)C₁₋₆alkyl, —NHC(O)C₁₋₆alkyl, —S(O)ₘNH₂, —S(O)ₘNHC₁₋₆alkyl, —S(O)ₘ(C₁₋₆alkyl)₂, aryl, heteroaryl and heterocyclyl
wherein m is 0,1, or 2,
or a pharmaceutically acceptable salt thereof, optionally in the form of a single stereoisomer or mixture of stereoisomers thereof.

10. The method of claim 1, where the insulin receptor-activating compound is a compound of Formula VI 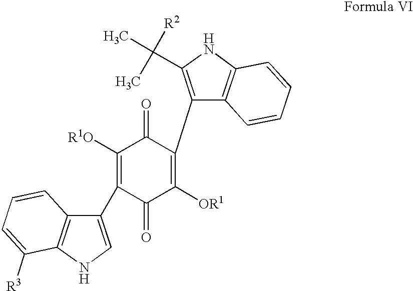
wherein
R¹ is hydrogen, or methyl
R² is —CH₂CH₃ or —CH═CH₂
R³ is —CH═CH—C(CH₃)═CH₂; CH₂—CH═C(CH₃)₂ or —CH₂—CH₂—CH═C(CH₃)₂,
or a pharmaceutically acceptable salt thereof, optionally in the form of a single stereoisomer or mixture of stereoisomers thereof

11. The method of claim 1, where the insulin receptor-activating compound is a compound of Formula VII 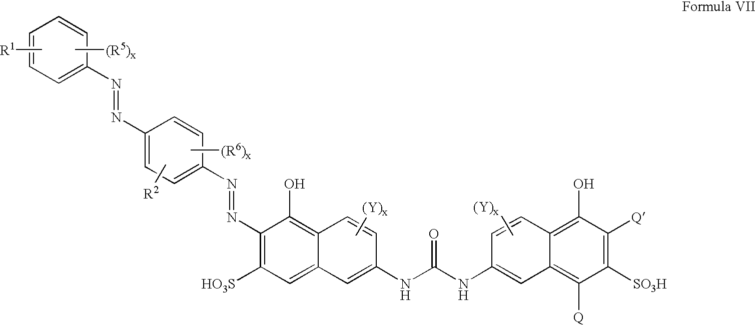
wherein
Q and Q′ are either hydrogen or 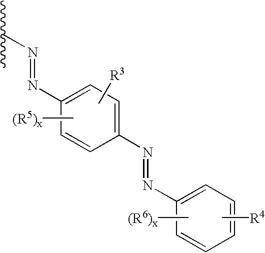
R¹ and R² are, independently, —SO₂NR⁷2, —C(O)NR⁷₂, —NR⁷SO₂R⁷, —SO₂OR⁷, —C(O)OR⁷, —PO₃ R⁷₂, or tetrazolyl,
R³ and R⁴ are, independently, —SO₂NR⁷₂, —C(O)NR⁷₂, —NR⁷C(O)R⁷, —SO₂OR⁷, —C(O)OR⁷, —PO₃R⁷₂, or tetrazolyl,
R⁵ and R⁶ are, independently, hydrogen, lower alkyl, substituted lower alkyl, cyano, halo, nitro, —SR⁸, —C(O)R⁸, —SO₂OR⁸, —OSO₂R⁸, —SO₂NR⁸₂, —NR⁸SO₂R⁸, —OC(O)R⁸, —C(O)OR⁸, —C(O)NR⁸₂, —NR⁸C(O)R⁸, —OR⁸, or —NR⁸₂,
each R⁷ and R⁸ is, independently, hydrogen, lower alkyl, substituted lower alkyl, aryl, substituted aryl, aryl(lower)alkyl, substituted aryl(lower)alkyl, heteroaryl(lower)alkyl, substituted heteroaryl-(lower)alkyl, heterocyclyl, substituted heterocyclyl, heteroaryl, or substituted heteroaryl,
each Y is a non-interfering substituent, and
each x is, independently, 0, 1 or 2,
or a pharmaceutically acceptable salt thereof, optionally in the form of a single stereoisomer or mixture of stereoisomers thereof.

12. The method of claim 1, further comprising administering a therapeutically effective amount of an additional formn of treatment for insulin resistance, hyperglycemia, diabetes, ketoacidosis, lipodystrophy, or hypertriglyceridemia.

13. The method of claim 12, wherein the therapeutically effective amount of the additional form of treatment when administered in combination with a compound of the invention is less than the amount of the additional form of treatment that would be therapeutically effective if delivered to the patient alone.

14. The method of claim 12, wherein the additional form of treatment is insulin.

15. The method of claim 12, wherein the additional form of treatment is an insulin analog.

16. The method of claim 14, wherein the therapeutically effective amount of insulin or insulin analog when administered in combination with a compound of the invention is less than the amount of insulin or insulin analog which would be therapeutically effective if delivered to the patient alone.

17. The method of claim 1, comprising administering two of the compounds of claims 3-11.

18. The method of claim 1, where the insulin receptor-activating compound is 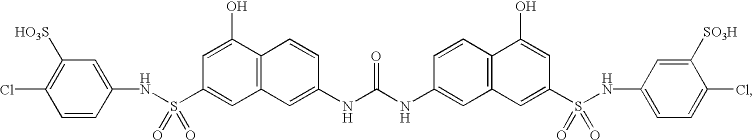
or a pharmaceutically acceptable salt thereof.

19. The method of claim 1, where the insulin receptor-activating compound is 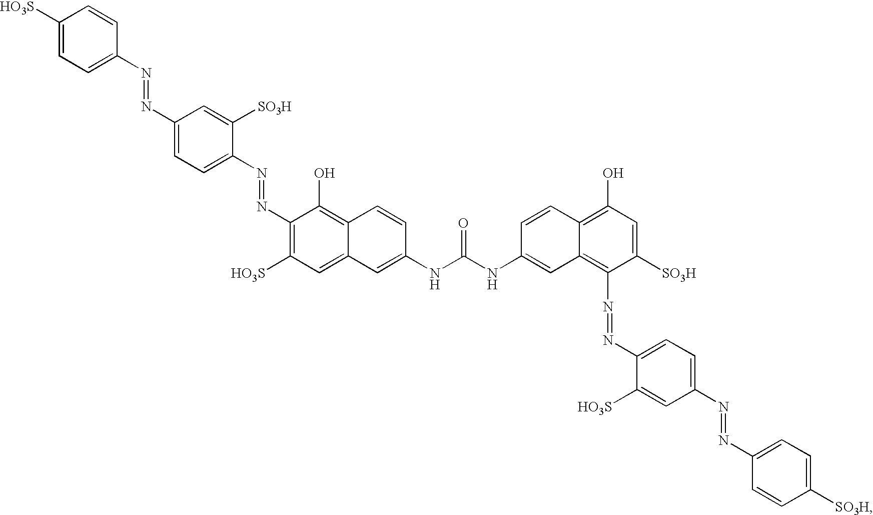
or a pharmaceutically acceptable salt thereof.

20. The method of claim 1, where the insulin receptor-activating compound is 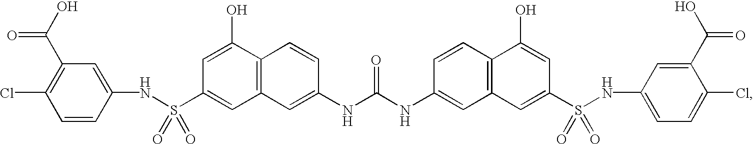
or a pharmaceutically acceptable salt thereof.
